(19) United States
(12) Patent Application Publication
(10) Pub. No.: US 2012/0095364 A1
(43) Pub. Date: Apr. 19, 2012

(54) Bolt Stop System For Use In Accessing Intracranial Space

(76) Inventor: Donald E. Bobo Sr., Fountain Valley, CA (US)

(21) Appl. No.: 13/148,937

(22) PCT Filed: Feb. 10, 2010
(86) PCT No.: PCT/US2010/023778

§ 371 Date: Dec. 13, 2011

Related U.S. Application Data
(60) Provisional application No. 61/151,404, filed on Feb. 10, 2009.

(51) Int. Cl. A61B 5/03; A61B 17/86

(52) U.S. Cl. 600/561; 606/304

(57) ABSTRACT

A bolt stop for use with an intracranial access bolt that assists in preventing penetration of the bolt into the skull, resists accidental rotation and movement of the bolt with in the skull, and facilitates healing of the scalp.

### RELATED APPLICATIONS

This application claims priority to International Patent Application No. PCT/US2010/023778, International Filing Date 10 Feb. 2010, entitled Bolt Stop System For Use In Accessing Intracranial Space, and to U.S. Provisional Application Ser. No. 61/151,404 filed Feb. 10, 2009, entitled Bolt Stop, the contents of both of which are incorporated herein in their entireties.

### FIELD OF THE INVENTION

The present invention relates to systems and methods for intracranial access and, more particularly to systems and methods for employing bolts for intracranial access.

### BACKGROUND OF THE INVENTION

There is a growing interest in the deployment of ventricular catheters and the data provided by probes placed in and proximate the brain. Such ventricular catheters and probes are deployed through an access point made in the skull of the patient. In certain prior art systems, the deployment of such ventricular catheters and probes is accomplished by first drilling a hole through the skull of the patient and then screwing a hollow bolt in to the drill hole. The shaft and the threads of the shaft of the hollow bolt are sized such that turning the head of the bolt causes the threads of the shaft to engage the sides of the drill hole and thereby bring the bolt further into the drill hole. The bolt must be screwed in the skull until a distal end of the bolt is approximately flush with the inner table of the skull. Skull thickness can vary from 3 to 20 millimeters. If the distal end of the bolt passes only partially through a thick skull, the probe cannot be deployed properly as it will depart the bolt at an angle and contact and/or bind on the burr or drill hole wall.

The torque required to screw in the bolt does not provide sufficient feedback that relates to the depth to which the bolt has been screwed. The operator must therefore count turns of the screw in order to determine the depth of the bolt in the skull. For example, the threads of a bolt may have a pitch such that for every complete revolution or turn of the bolt, the bolt advances one millimeter into the skull. Errors in counting turns of the bolt can present significant problems. If the turns are too few, probes deployed from the bolt will encounter the burr hole. If the turns are too many, the bolt will be screwed into brain tissue.

Once the bolt is deployed, a manifold having a guide tube is fixed to the bolt. The probe is then placed through an introducer having a curved distal end, and the introducer is passed through the guide tube. As the distal end of the introducer emerges from the guide tube, the curve in the distal end of the introducer reforms. The probe within the introducer is then directed away from the axis of the bolt and tissue disturbed by the introduction of a ventricular catheter. Once deployed, the probe and introducer curve reside in the brain angled away from the axis of the bolt. To avoid damage to the brain, the bolt must not be allowed to rotate once a probe is in place.

Additional details regarding systems and methods related to the use of a bolt stop in accordance with the present invention are disclosed in the U.S. application Ser. No. 10/855,159 to Bobo Sr. et al.; U.S. application Ser. No. 12/606,154 to Bobo Sr.; U.S. application Ser. No. 12/606,169 to Bobo Sr. et al.; U.S. Provisional Application No. 61/205,377 to Bobo et al.; U.S. Provisional Application No. 61/205,378 to Bobo et al.; International Application No. PCT/US09/62138 to Bobo Sr.; and International Application No. PCT/US10/21674 to Bobo et al., the contents of which are each herein incorporated in their entirety by reference.

### OBJECTS AND SUMMARY OF THE INVENTION

The devices and methods of the present invention address certain of the shortcomings in the prior art. The bolt stop of the present invention eliminates the need for the operator to count bolt rotations in order to determine the depth of the bolt in the skull. Accordingly, the present invention assists in preventing over or under engagement of the bolt within the skull. The present invention further provides increased resistance to accidental bolt rotation and bolt movement with in the skull. Finally, the present invention provides an improved interface between the bolt and the patient that facilitates healing of the disturbed scalp and skull.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1A is a front elevation view of a bolt according to certain embodiments of the present invention.

FIG. 1B is a plan view of a bolt according to certain embodiments of the present invention.

FIG. 2 is a series of cross-sectional views of bolt stops of various sizes according to certain embodiments of the present invention.

FIG. 3 is a perspective view of a bolt stop according to certain embodiments of the present invention.

FIG. 4 is a perspective view of a bolt stop according to certain embodiments of the present invention.

FIG. 5 is a front view of an assembled intracranial bolt, bolt stop, and insert assembly according to certain embodiments of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

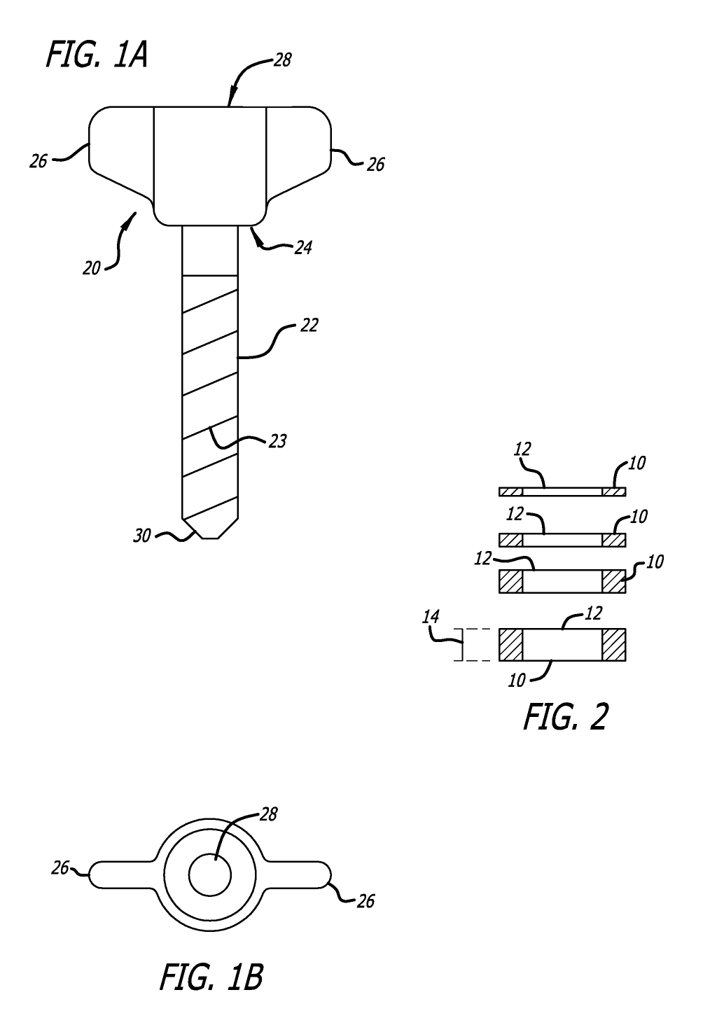

With reference to FIGS. 1-4, a bolt stop 10 according to the present invention is placed on the shaft 22 of the bolt 20 so as to stop the progression of a distal end 30 of the bolt 20 through the skull at a position substantially planar with an inner table or surface 42 of a skull 40. The bolt stop 10 of the present invention is used on intracranial access bolts 20 employed to direct ventricular catheters and probes placed into the skull and brain (not shown).

As shown in FIG. 2, in one embodiment of the present invention, the bolt stop 10 comprises one or more, preferably not more that 4, bodies of different heights 14. For example, the bolt stops 10 may be formed in the shape of a cylinder and have a height 14 in the range of 2 to 16 millimeters. Formed through the bolt stop 10 is lumen 12 that has a diameter slightly larger that an external diameter of a shaft 22 of a bolt 20, shown in FIG. 1 A. The bolt stop 10 may be formed of metal, alloy, silicone, or other polymeric material.

While the bolt stop 10 has been shown in FIGS. 1, 3, and 4 as having a cylindrical form, it is contemplated that other shapes, such as rectangles, triangles, and stars may be employed and that the bolt stop 10 may employ ends having different shapes. The stop bolt 10 may further employ supporting structures that extend from the sides or ends of the bolt stop 10 such as wings or buttresses that further aid in supporting and stabilizing the bolt 20.

In certain embodiments of the present invention, select surfaces of the bolt stop 10 are coated or otherwise treated with a material having therapeutic or preventive properties. For example, the surfaces of the bolt stop 10 that will contact the patient scalp and skull may be coated with an antibiotic material. Such a coating discourages microbe growth near the burr hole and on the face of the scalp through which the bolt 20 passes.

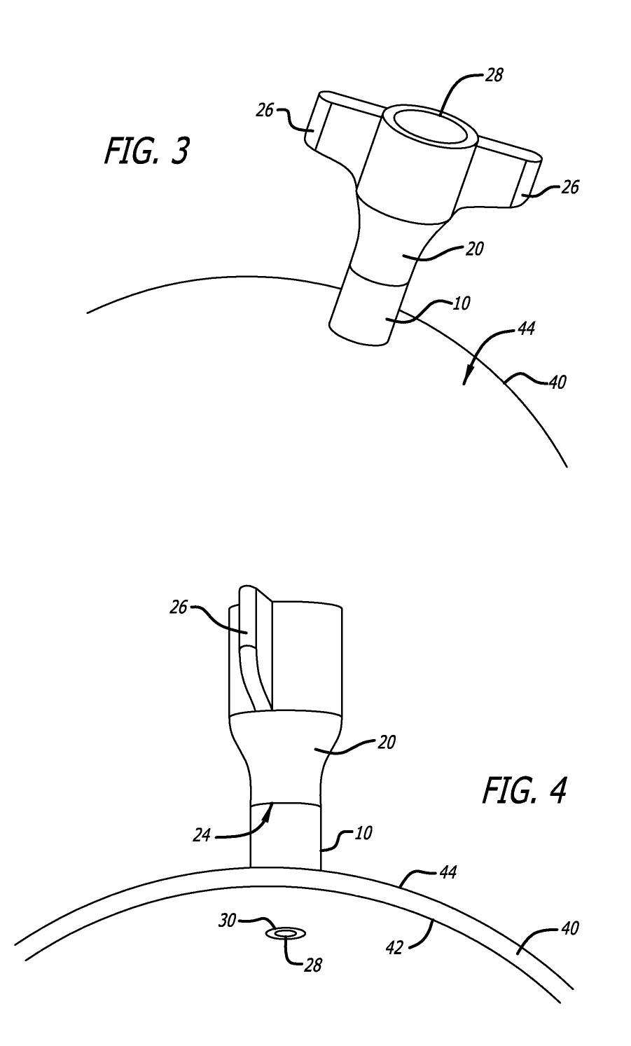

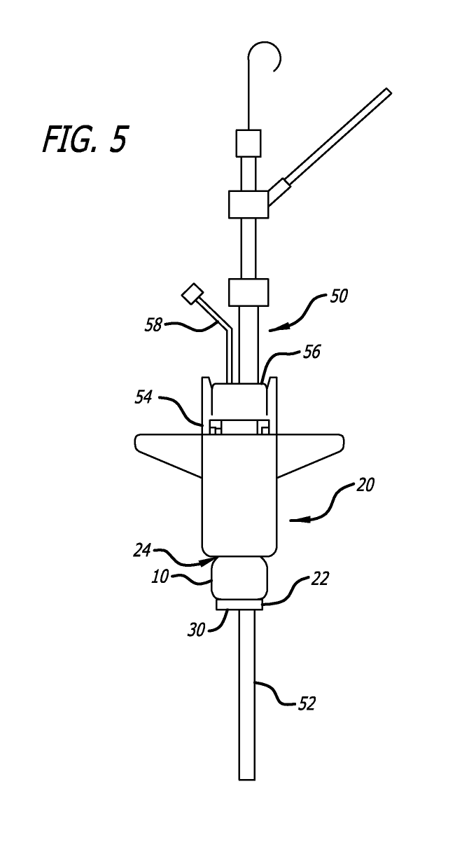

Turning to FIGS. 3 and 4, these figures show a bolt stop 10 and bolt 20 deployed within the skull of a patient. More particularly, FIG. 3 shows an exterior view of a patient skull in which a bolt 20 and bolt stop 10 have been deployed. FIG. 4 shows a cross-sectional view of a skull in which the distal end 30 of the bolt 20 is shown flush with the inner table 42 of the skull 40. Protruding from the outer table 44 of the skull 40 is the bolt stop 10 pinched between the proximal surface 24 of the bolt 20 and the outer table 44 of the skull 40.

Prior to deploying the bolt 20 and bolt stop 10, the thickness of a patient's skull 40 at the desired point of access is determined, e.g. through use of scanning and x-ray techniques employed in the art. Based on the determined thickness of the skull 40 and a known length of the shaft 22 of the bolt 20, the operator can determine the desired height 14 of the bolt stop 10 needed to prevent the distal end 30 of the shaft 22 from penetrating substantially beyond the inner table or surface of the patient's skull. For example, if the thickness of the skull 40 is determined to be 3 to 4 millimeters, the operator may employ a bolt stop 10 that has a height 14 of 16 millimeters in order to position a bolt 20 having a shaft 22 with a length of approximately 20 millimeters. Similarly, if the thickness of the patient's skull 40 is determined to be 9 to 10 millimeters, the operator may employ a bolt stop 10 that has a height 14 of approximately 10 millimeters in order to properly position a bolt 20 having a shaft 22 with a length of approximately 20 millimeters. The bolt stop 10 is selected such that, once the bolt stop 10 is moved up to the shaft 22 of the bolt 20 to abut the proximal surface 24 of the bolt, the length of the shaft 22 extending beyond the bolt stop 10 is equal to the thickness of the skull 40.

After the operator has determined the desired bolt stop 10 to utilize during the procedure, the shaft 22 of the bolt 20 is inserted through the lumen 12 of the bolt stop 10. The threads 23 of the shaft 22 of the bolt 20 are then screwed into a hole previously made in the patient's skull 40. As the bolt 20 is screwed into the patient's skull 40, the bolt stop 10 is displaced up the shaft 22 towards the proximal face 24 of the bolt 20. Once the distance between the proximal face 24 and a outer table or exterior surface 44 of the skull 40 equals the height 14 of the bolt stop 10, the bolt stop 10 resists further displacement of distal end 30 the into the skull. Stated alternatively, the bolt stop 10 becomes pinched between the proximal surface 24 of the bolt 20 and the outer table 44 of the skull 40.

After the bolt 20 and bolt stop 10 are deployed, depending upon the requirements of the intended procedure, the operator may utilize the bolt lumen 28 as a guide and access point for introducing therapeutic and diagnostic devices such as ventricular catheters and probes into the skull and brain of the patient.

In certain embodiments of the present invention, the bolt stop 10 is provided as a component in a kit comprising of a variety of bolt stops 10 having different heights, bolts 20 having various shaft 22 lengths, exterior diameters, and lumen 28 diameters, as well as other related components. It is further contemplated that the bolt stop 10 of the present invention comprises a component of an intracranial access kit incorporating some or all of the components described in the previously referenced U.S. and international patents and patent applications.

FIG. 5 shows an intracranial bolt 20 having a shaft 22 that is inserted through the bolt stop 10. The distal end 30 of the shaft 22 protrudes from one end of the bolt stop 10. Attached to the top of bolt 20 is an insert assembly 50 which comprises an insert body 56, bolt clamp 54, and a probe pigtail 58 through which an introducer is used to deploy a probe. Extending from the distal end 30 of the bolt 20 is a ventricular catheter 52. These components are disclosed in greater detail in the above referenced U.S. and international patents and patent applications.

The bolt stop 10 according to the present invention provides three advantageous functions. First, the bolt stop 10 provides an easier and safer means for deploying intracranial access bolts. For example, the bolt stop 10 eliminates the need for the operator to count bolt turns in order to determine the depth that the bolt 20 has penetrated into or through the skull. Eliminating the need to count turns assures that the distal end 30 of the bolt 20 is close enough to the inner table (bottom of burr hole) of the skull to allow probes to be deployed at an angle. Furthermore, the bolt stop 10 physically prevents the distal end 30 of the bolt 20 from penetrating beyond the inner table and potentially into the brain.

Second, the bolt stop 10 prevents inadvertent rotation of the bolt 20 because the friction fit of the bolt stop 10 between the skull 40 and the bolt 20 serves to lock the bolt 20 in place, i.e. prevents rotation of the bolt 20 within the skull. Once the bolt 20 is screwed against the bolt stop 10, the torque required to rotate the bolt 20 is such that it cannot be supplied by an unintentional contact with the bolt 20. The friction fit of the bolt stop 10 between the proximal surface 24 of the bolt 20 and the outer table 44 of the skull 40 further provides resistance and support against movement of the bolt 20 in a direction radial or perpendicular to the axis of the bolt 20. Significantly, this increased resistance to accidental rotation and movement is achieved without a corresponding increase in the size of the hole required to be made in the skull.

This feature is particularly advantageous in certain embodiments in which the bolt 20 employs a wing set 26 in order to torque the bolt 20. One of ordinary skill in the art will recognize that while bolt 20 employing the wing set 26 offers a significantly advantageous design, e.g. bolts employing wing sets do not require tools to torque; the wing set 26 may be inadvertently contacted such that the bolt 20 is rotated or moved within the skull 40. The present bolt stop 10 is operable to resist such inadvertent rotation or movement.

Third, the bolt stop 10 of the present invention provides an improved interface between the patient and the bolt 20. The bolt stop 10 provides a smooth, antimicrobial barrier around the skull burr hole and along the face of the scalp that would otherwise rests against the bolt. The antimicrobial coat on the bolt stop 10 protects the scalp and burr hole from contamination. Furthermore, the smooth surface of the bolt stop is less traumatic to the scalp than direct contact with the threads 23 of the shaft 22 of the bolt 20 as the bolt 20 is screwed in or out of the skull 40. The bolt stop 10 isolates the scalp from the sharp edges of the threads 23, edges which can tear and cut the tissue and result in bacteria being released from tissue. The bolt stop 10 thereby facilitates the healing of the scalp before and after bolt removal.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method for creating an intracranial access point comprising:
forming a hole in a skull;
inserting a threaded, hollow shaft through a lumen of at least one body;
engaging the threads of the hollow shaft with an interior surface of the hole in the skull by rotating the hollow shaft; and
rotating the hollow shaft until a proximal surface of the shaft pinches the body against a surface of the skull approximate the hole in the skull.

2. The method of claim 1 wherein the step of inserting a threaded, hollow shaft through a lumen of at least one body comprises inserting the threaded shaft through a lumen in a cylindrical body.

3. The method of claim 1 wherein the step of inserting a threaded, hollow shaft through a lumen of at least one body comprises inserting the threaded shaft through a lumen having a diameter that is slightly larger than an external diameter of the threaded shaft.

4. The method of claim 1 wherein the step of inserting a threaded, hollow shaft through a lumen of at least one body comprises inserting the threaded shaft through a lumen in a body, a portion of which is treated with a therapeutic agent.

5. The method of claim 4 wherein the therapeutic agent is antimicrobial.

6. The method of claim 1 wherein the step of inserting a threaded, hollow shaft through a lumen of at least one body comprises inserting the threaded shaft through a lumen formed by stacking a plurality of bodies having different heights.

7. The method of claim 1 wherein the step of rotating the hollow shaft until a proximal surface of the shaft pinches the body against a surface of the skull approximate the hole in the skill comprises substantially preventing rotation of the shaft when a distal end of the shaft is approximately planar with an inner table of the skull.

8. A method for deploying an intracranial bolt comprising:
surrounding a portion of a threaded shaft of an intracranial bolt with a body having a length less than a length of the shaft;
screwing the threaded shaft of the intracranial bolt into a hole in a skull; and
resisting the screwing of the threaded shaft of the intracranial bolt into the hole in the skull with the body when the threaded shaft has engaged the hole in the skull a distance approximately equal to a difference in the length of the body and the length of the shaft.

9. The method of claim 8 wherein the step of surrounding a portion of a threaded shaft of an intracranial bolt with a body having a length less than a length of the shaft comprises passing a portion of the threaded shaft through a lumen formed through the body.

10. The method of claim 8 wherein the step of surrounding a portion of a threaded shaft of an intracranial bolt with a body having a length less than a length of the shaft comprises passing a portion of the threaded shaft through a lumen formed through a cylindrical body.

11. The method of claim 8 further comprising the step of resisting a movement of the intracranial bolt in a direction perpendicular to an axis of the intracranial bolt.

12. The method of claim 11 wherein the step of resisting a movement of the intracranial bolt in a direction perpendicular to an axis of the intracranial bolt comprises supporting the shaft of intracranial bolt without increasing the diameter of the hole in the skull through which the intracranial bolt is screwed.

13. The method of claim 11 wherein the step of resisting a movement of the intracranial bolt in a direction perpendicular to an axis of the intracranial bolt comprises pinching the body between the skull and a proximal surface of the intracranial bolt.

14. The method of claim 8 wherein the step of screwing the threaded shaft of the intracranial bolt into a hole in a skull comprises rotating a wing set associated with the intracranial bolt.

15. The method of claim 8 wherein the step of resisting the screwing of the threaded shaft of the intracranial bolt into the hole in the skull when the threaded shaft has engaged the hole in the skull a distance approximately equal to a difference in the length of the body and the length of the shaft comprises pinching the body between the skull and a proximal surface of the intracranial bolt.

16. A method for deploying an intracranial probe comprising:
engaging a threaded shaft of an intracranial bolt with a hole in a skull by rotating the intracranial bolt;
preventing further rotation of the intracranial bolt when a distal end of the threaded shaft of the intracranial bolt is approximately planar with a interior surface of the skull; and
deploying a probe into the skull through a lumen formed axially through the intracranial bolt.

17. The method of claim 16 wherein the step of preventing further rotation of the intracranial bolt when a distal end of the threaded shaft of the intracranial bolt is approximately planar with a interior surface of the skull comprises interposing a body between a proximal surface of the intracranial bolt and the skull.

18. The method of claim 17 wherein the step of interposing a body between a proximal surface of the intracranial bolt and the skull comprises inserting a portion of the threaded shaft of the intracranial bolt through a lumen in the body.

19. The method of claim 16 further comprising the step of resisting a movement of the intracranial bolt in a direction perpendicular to an axis of the intracranial bolt.

20. The method of claim 19 wherein the step of resisting a movement of the intracranial bolt in a direction perpendicular to an axis of the intracranial bolt comprises pinching the body between the skull and a proximal surface of the intracranial bolt.

21. A system for accessing a cranial space comprising:
an intracranial bolt; and
a collection of bolt stops configured alone or in combination to resist an insertion of a distal end of the intracranial bolt substantially beyond an internal surface of a skull.

22. A system for measuring intracranial pressure comprising:
an intracranial bolt;
at least one bolt stop;
an insert assembly;
a ventricular catheter; and
at least one probe.
